(19) United States
(12) Patent Application Publication
(10) Pub. No.: US 2023/0255624 A1
(43) Pub. Date: Aug. 17, 2023

(54) SYSTEMS, DEVICES, AND METHODS FOR DELIVERING AND POSITIONING MAGNETIC ANASTOMOSIS COMPRESSION DEVICES FOR SUBSEQUENT FORMATION OF ANASTOMOSES

(71) Applicant: G.I. Windows, Inc., Westwood, MA (US)

(72) Inventors: Jeffrey M. Wallace, Pittsford, NY (US); Jonathan P. Boduch, Hanover, MA (US); Dane T. Seddon, Boston, MA (US)

(21) Appl. No.: 18/137,645

(22) Filed: Apr. 21, 2023

Related U.S. Application Data
(63) Continuation-in-part of application No. PCT/US2022/025338, filed on Apr. 19, 2022.
(60) Provisional application No. 63/177,200, filed on Apr. 20, 2021.

(51) Int. Cl. A61B 17/068; A61B 17/28

(52) U.S. Cl. CPC A61B 17/068; A61B 17/28; A61B 2017/00477

(57) ABSTRACT

The invention provides a system including a securement apparatus for providing improved placement and securement of magnetic compression devices at a desired target site so as to create anastomoses between tissues. The delivery apparatus is configured to be used in the placement and securement of magnetic compression devices that include one or more guide elements, such as sutures or wires, used for the positioning and/or manipulation of the devices once delivered to the target tissue, to achieve optimal placement and subsequent anastomosis formation.

### CROSS-REFERENCE TO RELATED APPLICATIONS

This patent application is a continuation-in-part of PCT Application No. PCT/US2022/025338 entitled SYSTEMS, DEVICES, AND METHODS FOR DELIVERING AND POSITIONING MAGNETIC ANASTOMOSIS COMPRESSION DEVICES FOR SUBSEQUENT FORMATION OF ANASTOMOSES filed Apr. 19, 2022 (Attorney Docket No. 121326-10902), which claims the benefit of United States Provisional Patent Application No. 63/177,200 entitled SYSTEMS, DEVICES, AND METHODS FOR DELIVERING AND POSITIONING MAGNETIC ANASTOMOSIS COMPRESSION DEVICES FOR SUBSEQUENT FORMATION OF ANASTOMOSES filed on Apr. 20, 2021 (Attorney Docket No. 121326-10901), each of which is hereby incorporated by reference herein in its entirety.

The subject matter of this patent application may be related to the subject matter of U.S. Pat. Application No. 17/108,840 entitled SYSTEMS, DEVICES, AND METHODS FOR FORMING ANASTOMOSES filed Dec. 1, 2020 (Attorney Docket No. 121326-11101), which is a continuation-in-part of, and therefore claims priority from, International Patent Application No. PCT/US2019/035202 having an International Filing Date of Jun. 3, 2019 (Attorney Docket No. 121326-11102), which claims the benefit of, and priority to, U.S. Provisional Application Serial No. 62/679,810, filed Jun. 2, 2018, U.S. Provisional Application Serial No. 62/798,809, filed Jan. 30, 2019, and U.S. Provisional Application Serial No. 62/809,354, filed Feb. 22, 2019, the contents of each of which are hereby incorporated by reference herein in their entireties.

### FIELD OF THE INVENTION

The invention relates to deployable magnetic compression devices, and, more particularly, to systems, devices, and methods for the deployment and positioning of magnetic compression devices at a desired site so as to improve the accuracy of anastomoses creation between tissues, organs, or the like.

### BACKGROUND

Bypasses of the gastroenterological (GI), cardiovascular, or urological systems are typically formed by cutting holes in tissues at two locations and joining the holes with sutures or staples. A bypass is typically placed to route fluids (e.g., blood, nutrients) between healthier portions of the system, while bypassing diseases or malfunctioning tissues. The procedure is typically invasive, and subjects a patient to risks such as bleeding, infection, pain, and adverse reaction to anesthesia. Additionally, a bypass created with sutures or staples can be complicated by post-operative leaks and adhesions. Leaks may result in infection or sepsis, while adhesions can result in complications such as bowel strangulation and obstruction. While traditional bypass procedures can be completed with an endoscope, laparoscope, or robot, it can be time consuming to join the holes cut into the tissues. Furthermore, such procedures require specialized expertise and equipment that is not available at many surgical facilities.

As an alternative to sutures or staples, surgeons can use mechanical couplings or magnets to create a compressive anastomosis between tissues. For example, compressive couplings or paired magnets can be delivered to tissues to be joined. Because of the strong compression, the tissue trapped between the couplings or magnets is cut off from its blood supply. Under these conditions, the tissue becomes necrotic and degenerates, and at the same time, new tissue grows around points of compression, e.g., on the edges of the coupling. With time, the coupling can be removed, leaving a healed anastomosis between the tissues.

Nonetheless, the difficulty of placing the magnets or couplings limits the locations that compressive anastomosis can be used. In most cases, the magnets or couplings have to be delivered as two separate assemblies, requiring either an open surgical field or a bulky delivery device. For example, existing magnetic compression devices are limited to structures small enough to be deployed with a delivery conduit e.g., an endoscopic instrument channel or laparoscopic port. When these smaller structures are used, the formed anastomosis is small and suffers from short-term patency. Furthermore, placement of the magnets or couplings can be imprecise, which can lead to anastomosis formation in locations that is undesirable or inaccurate.

Thus, there still remains a clinical need for reliable devices and minimally-invasive procedures that facilitate compression anastomosis formation between tissues in the human body.

### SUMMARY

Various embodiments of the invention provide improved devices and techniques for minimally-invasive formation of anastomoses within the body. Such devices and techniques facilitate faster and less-expensive treatments for chronic diseases such as obesity and diabetes. Such techniques also reduce the time and pain associated with palliative treatments for diseases such as cancers.

More specifically, the invention provides a securement apparatus for providing improved placement and securement of magnetic compression devices at a desired target site so as to create anastomoses between tissues. The securement apparatus is configured to be used in the placement and securement of magnetic compression devices that include one or more guide elements, such as sutures or wires, used for the positioning and/or manipulation of the devices once delivered to the target tissue, to achieve optimal placement and anastomosis formation.

For example, in an embodiment described herein, a securement apparatus for a compression anastomosis device comprises a button member shaped and sized to receive one or more guide elements. The guide elements may be coupled to a distal compression anastomosis device and the guide element may be capable of translating along the guide elements from a delivery device toward the distal anastomosis device and a target site. In an embodiment, the button member may then be centered relative to an enterotomy and the distal anastomosis device. In accordance with an embodiment, a plug member, shaped and sized to be received within the proximal end of the button member, is secured within the button member by friction fit, thereby securing one or more guide elements and the distal anastomosis device in place at a target site.

In various alternative embodiments, the plug member may be tapered in width such that the proximal end is wider than the distal end. The central aperture of the button member may also taper in width from the proximal-most opening toward the distal-most opening. The button member may also be shaped and/or sized to be positioned between a distal anastomosis device and a proximal anastomosis device. The button member may also be sized to have a diameter larger than that of the formed enterotomy. In various alternative embodiments, the proximal end of the button member may be sloped and/or dome shaped. The button member may also be configured to engage a portion of a first tissue wall of the hollow body of a patient. The button member and the plug member may both be made of medical-grade material.

In an alternative embodiment, a securement apparatus for securement of a compression anastomosis device comprises a deployable, self-assembling securement member. The self-assembling securement member may be capable of holding the position of a compression anastomosis device positioned at a target site relative to a tissue wall within a patient. In various embodiments, the securement member may be capable of capturing a formed enterotomy between the member’s distal and proximal ends and holding the compression anastomosis device in place until it is mated with a second compression anastomosis device.

In various alternative embodiments, the securement member is compressed within a delivery device, and is capable of expanding into a deployed configuration upon exiting the delivery device.

In some embodiments, the securement member may be a circular device, a balloon device, a “flower petal” device, a ratcheting device, a bulge tube device, or a spiral spring device.

In an alternative embodiment, an apparatus for assisting with the placement and securement of a compression anastomosis device comprises a limiting member, capable in some embodiments of capturing a pair of compression anastomosis devices between the member’s proximal and distal ends. In some embodiments, the limiting member may be rigidly attached to a distal anastomosis device. In some embodiments, the limiting member may only move in the compressive direction, thereby preventing any increase in distance between the proximal and distal anastomosis devices.

In various alternative embodiments, the limiting member may be capable of being compressed in a delivery device, and able to expand into a deployed configuration upon exiting the delivery device. In some embodiments, the limiting member may be capable of compressing back into a compressed configuration in order to be retracted into the delivery device for removal from a patient.

In various alternative embodiments, the limiting member may be a balloon, a wire array, a “flower petal”, a bulge tube, a ratcheting device, a suspender device, and/or a rebar tie device.

### BRIEF DESCRIPTION OF THE DRAWINGS

Features and advantages of the claimed subject matter will be apparent from the following detailed description of embodiments consistent therewith, which description should be considered with reference to the accompanying drawings, wherein:

FIG. 1 is a schematic illustration of an anastomosis formation system consistent with the present disclosure.

FIG. 2 shows several potential anatomical targets for anastomosis formation, where arrow A is stomach to small intestine, arrow B is small intestine to large intestine, arrow C is small intestine to small intestine, arrow D is large intestine to large intestine, and arrow E is stomach to large intestine.

FIG. 3 shows an exemplary magnetic anastomosis device delivered through an endoscope instrument channel such that the individual magnet segments self-assemble into a larger magnetic structure-in this particular case, an octagon.

FIG. 4A depicts two magnetic anastomosis devices attracting each other through tissue. As shown, the devices each comprise eight magnetic segments, however alternate configurations are possible. Once the two devices mate, the tissue that is trapped between the devices will necrose, causing an anastomosis to form. Alternatively, the tissue bound by the devices may be perforated after the devices mate to create an immediate anastomosis.

FIG. 4B shows the two magnetic anastomosis devices coupled together by magnetic attraction, capturing the intervening tissue. In some instances, the endoscope can be used to cut through the circumscribed tissue.

FIG. 5A shows the needle delivering a first magnetic device into a first portion of the hollow body at the target site.

FIG. 5B shows subsequent deployment to of a second magnetic device into a second portion of the hollow body adjacent to the target site.

FIG. 6A shows endoscopic ultrasound guided needle delivery of a magnet assembly into the gallbladder which then couples with a second magnet assembly in the stomach or duodenum as shown in FIG. 6B.

FIG. 7 illustrates a single guide element for deploying and manipulating a magnetic anastomosis device.

FIGS. 8A, 8B, 8C, 8D, 8E, and 8F each depict the deployment of the self-closing magnetic anastomosis device with a plurality of guide elements.

FIGS. 9A and 9B illustrate the formation of an enterotomy in at least a first portion of the first tissue at the target site via a needle of the access device.

FIG. 9C illustrates delivery and self-assembling of the first magnetic anastomosis device relative to the first tissue.

FIGS. 9D-9G illustrates positioning of the first magnetic anastomosis device via manipulation of the guide elements coupled thereto and securing of the first magnetic anastomosis device into place via the securement apparatus; and

FIGS. 9H and 9I illustrate delivery and positioning of a second magnetic anastomosis device to a second tissue adjacent the first tissue at the target site and subsequent mating of the first and second magnetic anastomosis devices to one another by way of magnetic attraction forces to thereby cause compression of the tissues for subsequent anastomosis formation.

FIG. 10 shows a balloon securement mechanism that can be inflated from the side of the enterotomy opposite the compression anastomosis device, in accordance with one exemplary embodiment.

FIG. 11 shows a circular securement mechanism that can self-deploy from the side of the enterotomy opposite the compression anastomosis device, in accordance with one exemplary embodiment.

FIG. 12 shows a “flower petal” securement mechanism that can be deployed from the side of the enterotomy opposite the compression anastomosis device, in accordance with one exemplary embodiment.

FIG. 13 shows a securement mechanism that can be deployed from the side of the enterotomy opposite the compression anastomosis device and also including a ratcheting mechanism that only allows movement in the compressive direction, preventing any increase in the distance between the compression anastomosis device and the securement mechanism, in accordance with one exemplary embodiment.

FIG. 14 shows a bulge tube securement mechanism that expands on both sides of the enterotomy by pulling the two ends toward one another and thereby to secure the compression anastomosis device at the site of the enterotomy, in accordance with one exemplary embodiment.

FIG. 15 shows a spiral spring securement mechanism that can deployed from the side of the enterotomy opposite the compression anastomosis device, in accordance with one exemplary embodiment.

FIG. 16 shows a balloon limiting mechanism, in accordance with one exemplary embodiment.

FIG. 17 shows a wire array limiting mechanism, in accordance with one exemplary embodiment.

FIG. 18 shows a “flower petal” limiting mechanism, in accordance with one exemplary embodiment.

FIG. 19 shows a bulge tube limiting mechanism, in accordance with one exemplary embodiment.

FIG. 20 shows a limiting mechanism that also includes a ratcheting mechanism that only allows movement in the compressive direction, preventing any increase in the distance between the compression anastomosis device and the securement mechanism, in accordance with one exemplary embodiment.

FIG. 21 shows a limiting mechanism with suspenders, in accordance with one exemplary embodiment.

FIG. 22 shows a rebar tie limiting mechanism, in accordance with one exemplary embodiment.

For a thorough understanding of the present disclosure, reference should be made to the following detailed description, including the appended claims, in connection with the above-described drawings. Although the present disclosure is described in connection with exemplary embodiments, the disclosure is not intended to be limited to the specific forms set forth herein. It is understood that various omissions and substitutions of equivalents are contemplated as circumstances may suggest or render expedient.

### DETAILED DESCRIPTION

The invention provides a system for providing improved placement of magnetic compression devices at a desired target site so as to create anastomoses between tissues. The system generally includes an access device configured to be provided within a hollow body of a patient and assist in the formation of an anastomosis at a target site (a desired anatomical location) within the hollow body for formation of an anastomosis between a first portion of tissue of the hollow body at the target site and a second portion of tissue of the hollow body. The access device is configured to provide access to the first and second portions of tissue of the hollow body and further deliver and position first and second implantable magnetic anastomosis devices relative to the first and second portions of tissue or adjacent tissue for the formation of an anastomosis between tissues at the target site. The first and second implantable magnetic anastomosis devices are configured to be magnetically attracted to one another through a defined tissue area of the combined thickness of a wall of the tissues at the target site and exert compressive forces on the defined area to form the anastomosis.

The system further includes a securement apparatus for providing improved placement and securement of magnetic compression devices at a desired target site so as to create anastomoses between tissues. The securement apparatus is configured to be used in the placement and securement of magnetic compression devices that include one or more guide elements, such as sutures or wires, used for the positioning and/or manipulation of the devices once delivered to the target tissue, to achieve optimal placement and anastomosis formation. For example, in the embodiments described herein, at least one of a pair of self-assembling magnetic anastomosis devices (which are configured to couple together by way of magnetic attraction to thereby capture intervening tissue therebetween, which will subsequently necrose, causing an anastomosis to form) includes one or more guide elements coupled thereto. Each guide element may include a distal end coupled to a respective portion of the anastomosis device, and a proximal end that can be manipulated (i.e., increased or decreased tension) to thereby manipulate the positioning and orientation of the anastomosis device once it has self-assembled into the pre-determined shape (i.e., a polygon).

Upon delivery and deployment of the first magnetic anastomosis device at the target tissue site, the one or more guide elements may generally extend from the anastomosis device, through the enterotomy formed via the delivery needle and through the hollow body of the patient back towards the access device and potentially to an exterior portion of the patient. For example, in the event that an anastomosis is to be formed between the stomach and the gallbladder (or duodenum and gallbladder), the first magnetic anastomosis device is placed and deployed within the gallbladder, wherein guide elements coupled thereto may then remain attached to the anastomosis device and extend through the GI tract back towards the access device and potentially to the exterior of the patient (i.e., extend from the anastomosis device, through the access site into the gallbladder, into the stomach, up the esophagus, and out of the mouth).

The securement apparatus of the present invention is configured to secure the first magnetic anastomosis device in place at the target site, thereby ensuring that the first magnetic anastomosis device remains in position relative to the target site while the surgeon delivers and deploys of the second magnetic anastomosis device at the target site. For example, the securement apparatus generally includes a button member including a body having a distal end, an opposing proximal end, and a central aperture or through hole extending from the distal end to the proximal end. The aperture is shaped and/or sized to receive the one or more guide elements therethrough, such that the button member can generally translate along the length of the guide element extending from the first magnetic anastomosis device and through the enterotomy.

For example, once the first magnetic anastomosis device is deployed at the target site (i.e., passed through an enterotomy formed through a first tissue at the target site and self-assembled into the predefined shape), the guide elements, which extend back through the enterotomy, can be threaded through the central aperture of the body of the button member. A surgeon then need only pull the guide elements taught so as to draw the deployed first magnetic anastomosis device into engagement with a first side of the first tissue, essentially centering the first magnetic anastomosis device relative to the enterotomy. The button member may then translate along a length of the guide elements (passing through the central aperture) until the distal end of the body engages an opposing side of the first tissue, such that the button member is centered relative to the enterotomy and the opposing first magnetic anastomosis device.

The securement apparatus further includes a plug member including a distal end shaped and/or sized to be received within at least a proximal portion of the central aperture of the button member and retained therein via a friction fit. For example, the plug member may taper in width from a proximal end to the distal end (i.e., greater width at proximal end and less width at the distal end). Accordingly, upon pulling the guide elements taught and positioning the button member into engagement with the first tissue, a surgeon can drive the plug member into the central aperture of the button, which essentially secures the button member and the opposing first magnetic anastomosis device in position relative to the target site (i.e., fixed on opposing sides of the first tissue wall). More specifically, the plug member secures the button member and guide elements, which are passing through the central aperture of the button member, to one another via the friction fit. In some alternative embodiments, the plug member may be replaced with any type of friction fit device to hold the guide elements in place within the button member. The guide elements can be cut and removed once the securement apparatus and the opposing first magnetic anastomosis device are essentially fixed in place along the tissue wall.

Upon fixing the first magnetic anastomosis device in place, via the securement apparatus, the second magnetic anastomosis device can be subsequently delivered and deployed at the target site via the access device. Upon reaching the target tissue site, the first and second magnetic anastomosis devices will be magnetically attracted to one another through a defined tissue area of the combined thickness of a wall of the tissues at the target site (i.e., gallbladder wall and stomach wall, upper and lower intestinal walls, etc.) and exert compressive forces on the defined area to form the anastomosis. In some alternative embodiments, the securement apparatus may engage with both the first and second tissues, so as to compress both tissues against the first anastomosis device, securing the first anastomosis device in place to be accurately paired with the second anastomosis device.

Accordingly, the securement apparatus of the present disclosure provides improved placement and securing of at least a first magnetic compression device at a desired target site to thereby improve subsequent positioning and placement of a second corresponding magnetic anastomosis device at the target site, so as to create accurate anastomoses between tissues. The securement apparatus is configured to utilize guide elements from a first anastomosis device, already deployed and placed at the desired target tissue site, as a means for essentially fixing the first magnetic anastomosis device in place, thereby ensuring a high degree of precision and improved placement of a subsequent second corresponding magnetic anastomosis device to be coupled to the first device (i.e., ensuring that the anastomosis devices are placed and coupled to one another at the intended target site) to achieve optimal placement and anastomosis formation.

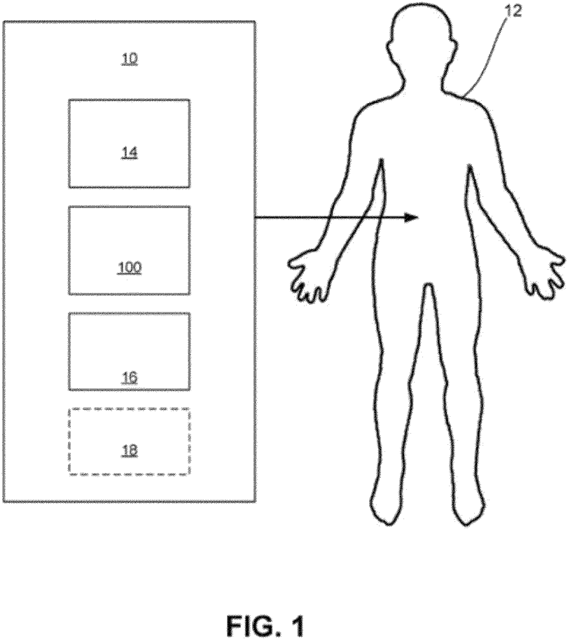

FIG. 1 is a schematic illustration of an anastomosis formation system 10 for providing improved placement of magnetic anastomosis devices 16 at a desired site so as to improve the accuracy of anastomoses creation between tissues within a patient 12. The system 10 generally includes an access device 14, a securement apparatus 100, magnetic anastomosis devices 16, and an imaging modality 20.

The access device 14 may generally include a scope, including, but not limited to, an endoscope, laparoscope, catheter, trocar, robotic device, or other delivery device. Placement of the anastomosis devices may also occur in an open field. For most applications described herein, the access device 14 is an endoscope, including a delivery needle 28 configured to deliver the magnetic anastomosis devices 16a, 16b. Accordingly, the system 10 of the present disclosure relies on a single endoscope 14 for the delivery of the two magnetic devices 16a, 16b. As will be described in greater detail herein, a surgeon may advance the endoscope 14 within a hollow body of the patient 12 and position the endoscope 14 at the desired anatomical location for formation of the anastomosis based on a visual depiction of the location of the target site as provided by the imaging modality 20. For example, the imaging modality 20 may include a display in which an image, or other visual depiction, is displayed to the surgeon illustrating a target site when performing a medical imaging procedure, including, but not limited to, ultrasound (US), wavelength detection, X-ray-based imaging, illumination, computed tomography (CT), radiography, and fluoroscopy, or a combination thereof. The surgeon may then rely on such a visual depiction when advancing the endoscope through the hollow body so as to position the access device 14 at a portion of tissue adjacent to the other portion of tissue at the target site, thereby ensuring the placement of the magnetic devices 16a, 16b is accurate.

It should be noted that the hollow body through which the access device 14 may pass includes, but is not limited to, the stomach, gallbladder, pancreas, duodenum, small intestine, large intestine, bowel, vasculature, including veins and arteries, or the like.

In some embodiments, the self-assembling magnetic devices 16a, 16b are used to create a bypass in the gastrointestinal tract. Such bypasses can be used for the treatment of a cancerous obstruction, weight loss or bariatrics, or even treatment of diabetes and metabolic disease (i.e. metabolic surgery).

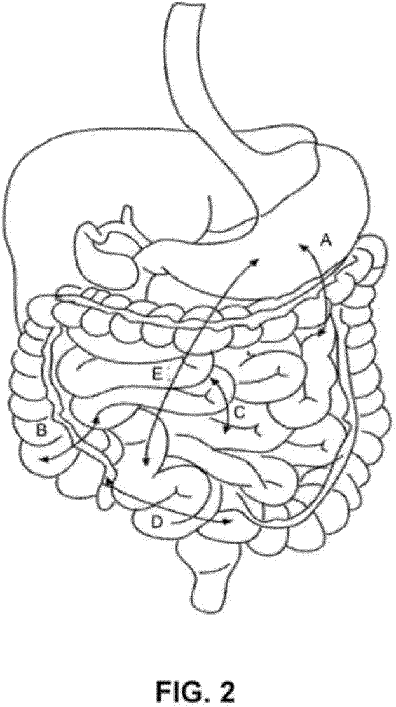

FIG. 2 illustrates the variety of gastrointestinal anastomotic targets that may be addressed with the devices of the invention, such targets include stomach to small intestine (A), stomach to large intestine (E), small intestine to small intestine (C), small intestine to large intestine (B), and large intestine to large intestine (D). Accordingly, the invention provides improved devices and techniques for minimally-invasive formation of anastomoses within the body, e.g., the gastrointestinal tract. Such devices and techniques facilitate faster and less-expensive treatments for chronic diseases such as obesity and diabetes. Such techniques also reduce the time and pain associated with palliative treatments for diseases such as cancers, such as stomach or colon cancer.

For example, if the hollow body through which the access device 14 may pass is a bowel of the patient, the first portion may be a distal portion of the bowel and the second portion may be a proximal portion of the bowel. The bowel includes any segment of the alimentary canal extending from the pyloric sphincter of the stomach to the anus. In some embodiments, an anastomosis is formed to bypass diseased, mal-formed, or dysfunctional tissues. In some embodiments, an anastomosis is formed to alter the “normal” digestive process in an effort to diminish or prevent other diseases, such as diabetes, hypertension, autoimmune, or musculoskeletal disease. It should be noted that the system may be used for the formation of an anastomosis between a first portion of tissue of the hollow body at the target site and an adjacent tissue of a second hollow body (e.g., portal between the stomach and the gallbladder, the duodenum and the gallbladder, stomach to small intestine, small intestine to large intestine, stomach to large intestine, etc.).

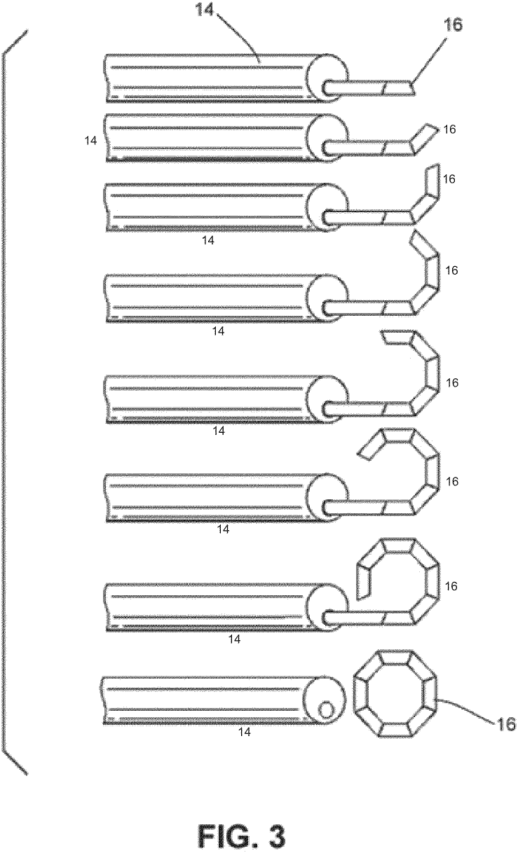

In an endoscopic procedure, the self-assembling magnetic devices 16 can be delivered using a single endoscope 14. Deployment of a magnetic device 16 is generally illustrated in FIG. 3. As shown, exemplary magnetic anastomosis devices 16 may be delivered through an endoscope 14 such that individual magnet segments self-assemble into a larger magnetic structure-in this particular case, an octagon. When used with the techniques described herein, the devices 16 allow for the delivery of a larger magnetic structures than would otherwise be possible via a small delivery conduit 14, such as in a standard endoscope, if the devices were deployed as a completed assembly. Larger magnet structures, in turn, allow for the creation of larger anastomoses that are more robust, and achieve greater surgical success. Because the magnetic devices 16 are radiopaque and echogenic, the devices can be positioned using fluoroscopy, direct visualization (trans-illumination or tissue indentation), and ultrasound, e.g., endoscopic ultrasound. The devices 16 can also be ornamented with radiopaque paint or other markers to help identify the polarity of the devices during placement.

The magnetic anastomosis devices 16 of the invention generally comprise magnetic segments that can assume a delivery conformation and a deployed configuration. The delivery configuration is typically linear so that the device can be delivered to a tissue via a laparoscopic “keyhole” incision or with delivery via a natural pathway, e.g., via the esophagus, with an endoscope 14 or similar device. Additionally, the delivery conformation is typically somewhat flexible so that the device can be guided through various curves in the body. Once the device 16 is delivered, the device 16 will assume a deployed configuration of the desired shape and size by converting from the delivery configuration to the deployed configuration automatically. The self-conversion from the delivery configuration to the deployment configuration is directed by coupling structures that cause the magnetic segments to move in the desired way without intervention. Exemplary self-assembling magnetic anastomosis devices 16, such as self-closing, self-opening, and the like, are described in U.S. Pat. No. 8,870,898, U.S. Pat. No. 8,870,899, U.S. Pat. No. 9,763,664, and U.S. Pat. Application No. 14/805,916, filed Jul. 22, 2015, the contents of each of which are incorporated by reference herein in their entirety.

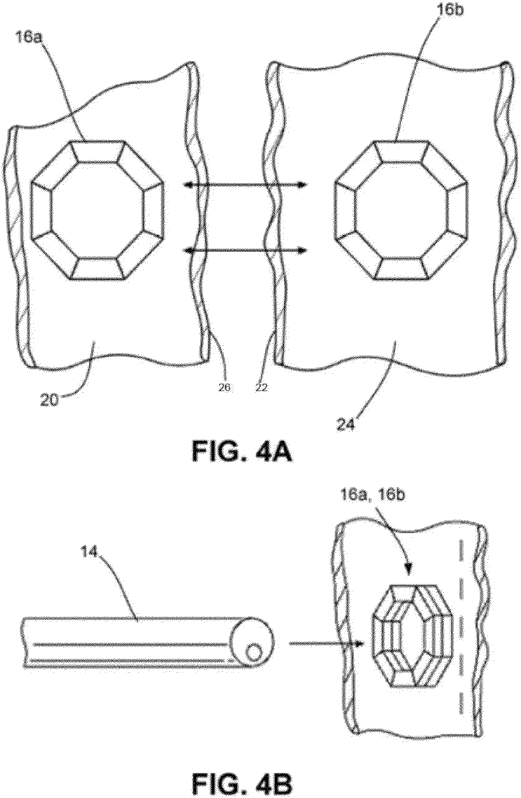

In general, as shown in FIG. 4A, a magnetic anastomosis procedure involves placing a first and a second magnetic structures 16a, 16b adjacent to first and second portions 20, 24 of tissues 26, 22, respectively, thus causing the tissues 22 and 26 to come together. Once the two devices 16a, 16b are brought into proximity, the magnetic structures 16a, 16b mate and bring the tissues 22, 26 together. With time, an anastomosis of the size and shape of the devices 16a, 16b will form and the devices will fall away from the tissue. In particular, the tissues 22, 26 circumscribed by the devices will be allowed to necrose and degrade, providing an opening between the tissues.

Alternatively, because the mated devices 16a and 16b create enough compressive force to stop the blood flow to the tissues 22, 26 trapped between the devices, a surgeon may create an anastomosis by making an incision in the tissues 22, 26 circumscribed by the devices, as shown in FIG. 4B.

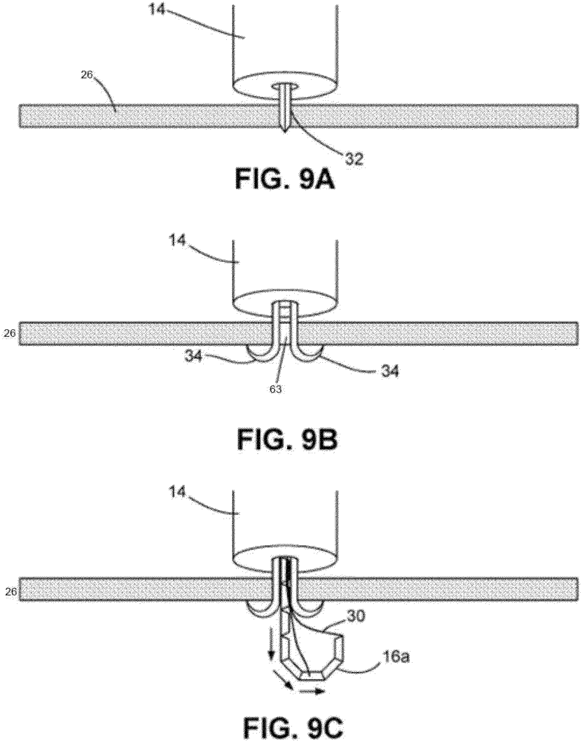

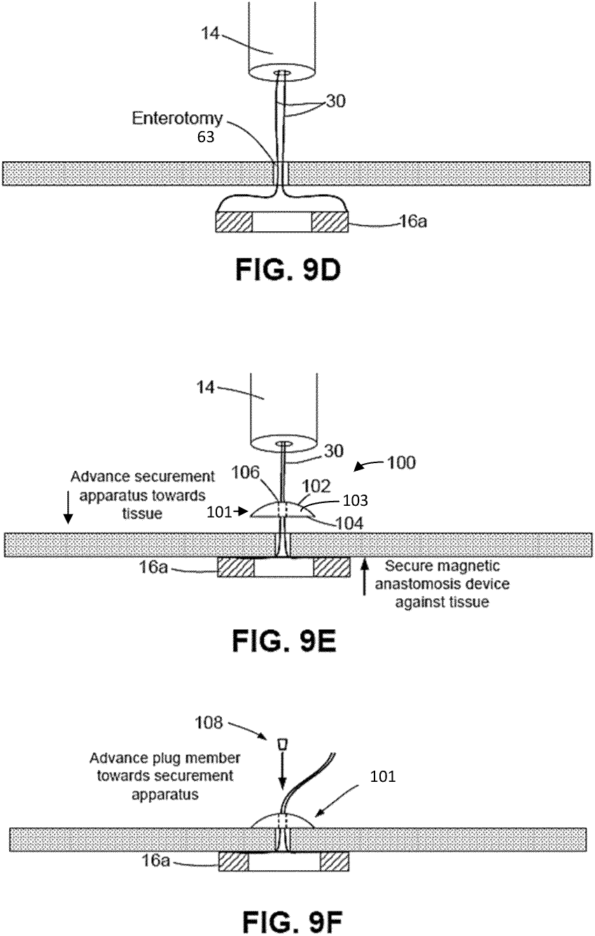

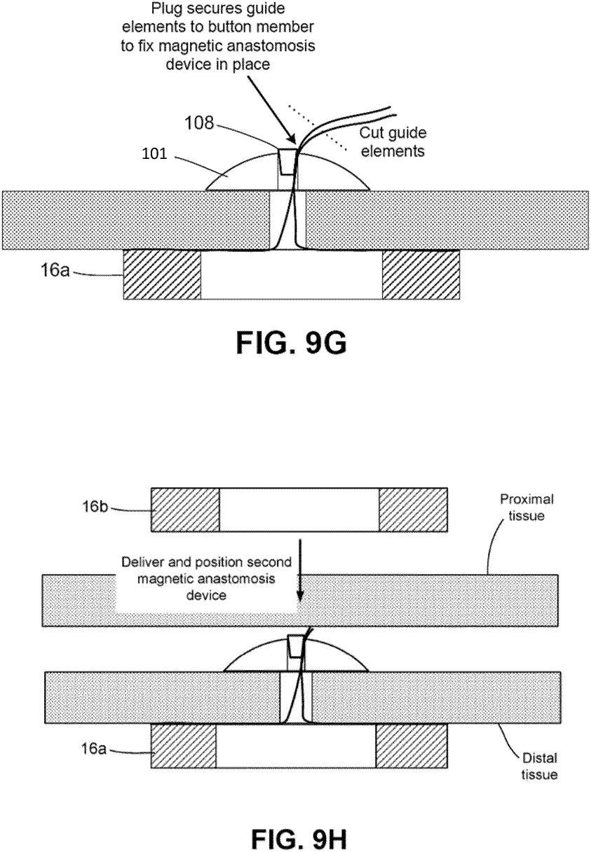

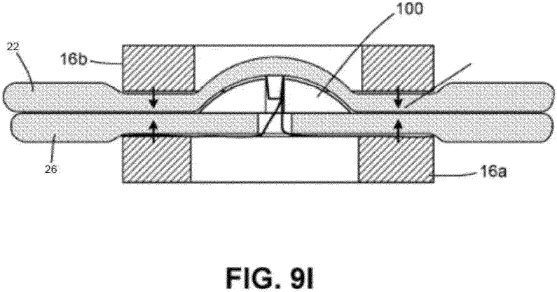

In yet another embodiment, as will be described in greater detail herein, and shown in FIGS. 9A-9I, a surgeon may first cut into, or pierce, the tissues 22, 26, and then deliver device 16a into a portion 20 of the hollow body so as to place device 16a around the incision on tissue 26. The surgeon may then place device 16b into portion 24 of the hollow body so as to deliver device 16b around the incision on tissue 22, and then allow the devices 16a and 16b to couple to one another, so that the devices 16a, 16b circumscribe the incision. As before, once the devices 16a, 16b mate, the blood flow to the incision is quickly cut off.

While the figures and structures of the disclosure are primarily concerned with annular or polygonal structures, it is to be understood that the delivery and construction techniques described herein can be used to make a variety of deployable magnetic structures. For example, self-assembling magnets can re-assemble into a polygonal structure such as a circle, ellipse, square, hexagon, octagon, decagon, or other geometric structure creating a closed loop. The devices 16a, 16b may additionally include handles, suture loops, barbs, and protrusions, as needed to achieve the desired performance and to make delivery (and removal) easier.

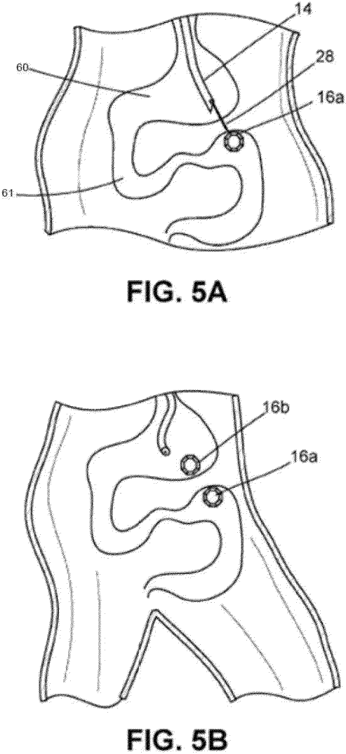

As previously described, the self-assembling magnetic anastomosis devices 16a, 16b can be delivered to the target site via the access device 14. For example, as shown in FIG. 5A, the access device 14 may include a delivery needle 28 (e.g., an aspiration needle) used to deliver the first magnetic anastomosis device 16a into the lower small intestine (through the puncture), which is then followed by deployment to of a second magnetic device 16b into the upper small intestine at a location on the tissue adjacent to the target site (shown in FIG. 5B). It should be noted that the delivery can be guided with fluoroscopy or endoscopic ultrasound. Following self-assembly, these small intestine magnetic devices 16a, 16b couple to one another (e.g., magnetically attracted to one another) through a defined tissue area of the combined thickness of a wall of the tissues at the target site and exert compressive forces on the defined area to form the anastomosis.

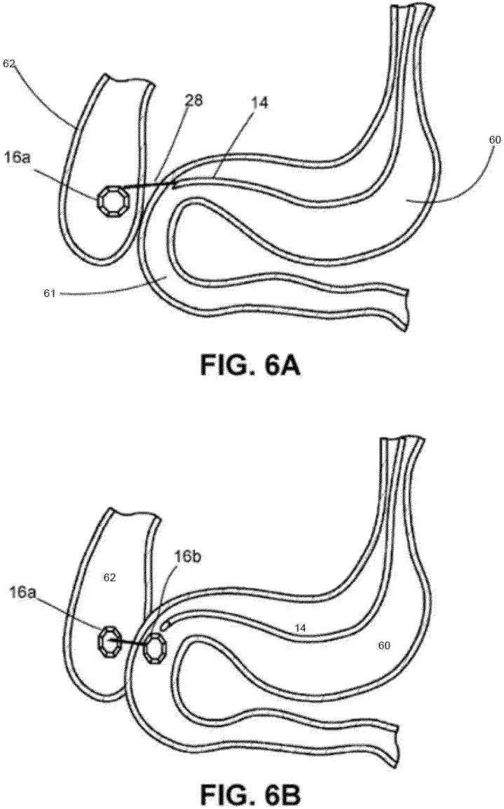

FIG. 6A shows endoscopic ultrasound guided needle delivery of a magnet assembly 16a into the gallbladder 62 which then couples with a second magnet assembly 16b in the stomach 60 or duodenum 61 as shown in FIG. 6B. Accordingly, the described procedures may also be used with procedures that remove or block the bypassed tissues. For example, endoscopic ultrasound (EUS) can be used to facilitate guided transgastric or transduodenal access into the gallbladder 62 for placement of a self-assembling magnetic anastomosis device 16a. Once gallbladder 62 access is obtained, various strategies can be employed to maintain a patent portal between the stomach 60 and the gallbladder 62 or the duodenum 61 and the gallbladder 62. In another embodiment, gallstones can be endoscopically retrieved and fluid drained. For example, using the described methods, an anastomosis can be created between the gallbladder 62 and the stomach 60. Once the gallbladder 62 is accessed in a transgastric or transduodenal fashion, the gallstones can be removed. Furthermore, the gallbladder mucosa can be ablated using any number of modalities, including but not limited to argon plasma coagulation (APC), photodynamic therapy (PDT), sclerosant (e.g. ethanolamine or ethanol).

As previously described, the securement apparatus 100 provides improved placement and securement of magnetic compression devices 16a, 16b at a desired target site so as to create anastomoses between tissues 22, 26. The securement apparatus 100 is configured to be used in the placement and securement of magnetic compression devices 16a, 16b that include one or more guide elements 30, such as sutures or wires, used for the positioning and/or manipulation of the devices once delivered to the target tissue, to achieve optimal placement and anastomosis formation.

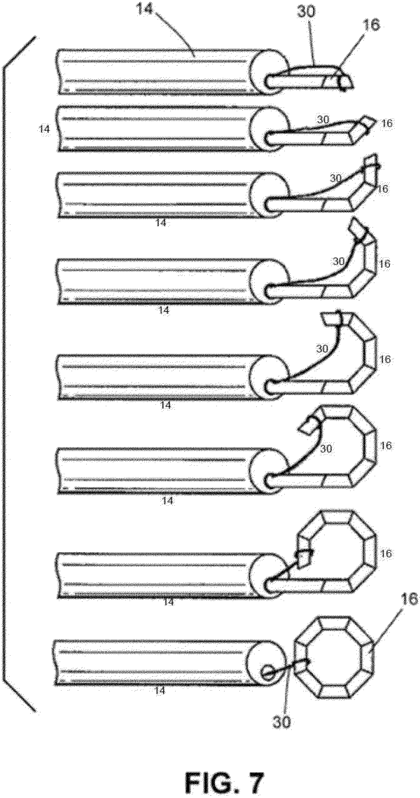

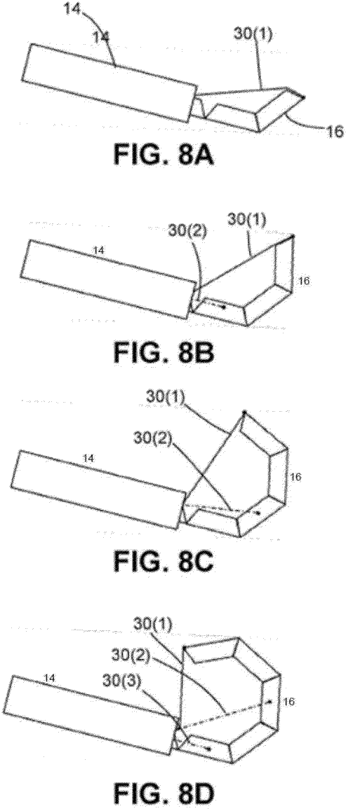

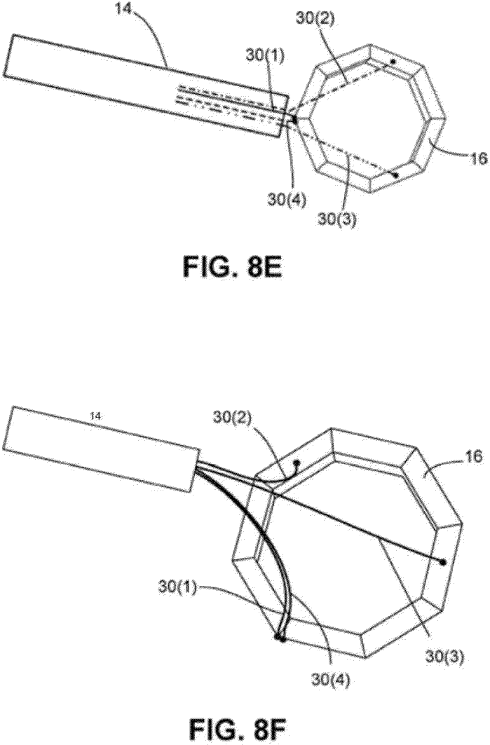

FIG. 7 illustrates a single guide element 30 for deploying and manipulating a magnetic anastomosis device 16. For example, once the self-assembling magnetic device 16 has been delivered to a tissue, it is beneficial to be able to manipulate the location of the device 16. While the device 16 can be manipulated with conventional tools such as forceps, it is often simpler to manipulate the location of the deployed device 16 with a guide element 30, such as a suture or wire. As shown in FIGS. 7 and 8A-8F, a variety of attachment points can be used to provide control over the location and deployment of a self-assembling magnetic anastomosis device 16.

For example, as shown in FIG. 7, the guide element 30 may be coupled to a single distal segment 16 such that, upon self-assembly, the single distal segment 16 results in an attachment point that provides translational freedom of movement. It is also notable that the configuration shown in FIG. 7 also allows a closing force to be applied to the distal-most segment. That is, in the event that one or more segments should become entangled with tissue, or otherwise prevented from self-assembling, a proximal pulling force with the guide element 30 can help the device 16 to complete self-assembly. Once self-assembly is completed, the device 16 can be positioned with the guide element 30 to be mated with another device (not shown) to form an anastomosis, as described above. While it is not shown in FIG. 7, it is envisioned that additional structures, such as a solid pusher or a guide tube can be used to deploy the device 16 in the desired location.

The guide element 30 can be fabricated from a variety of materials to achieve the desired mechanical properties and bio-compatibility. The guide element 30 may be constructed from metal, e.g., wire, e.g., stainless steel wire, or nickel alloy wire. The guide element may be constructed from natural fibers, such as cotton or an animal product. The guide element may be constructed from polymers, such as biodegradable polymers, such as polymers including repeating lactic acid, lactone, or glycolic acid units, such as polylactic acid (PLA). The guide element may also be constructed from high-tensile strength polymers, such as Tyvek™ (high-density polyethylene fibers) or Kevlar™ (para-aramid fibers). In an embodiment, guide element 30 is constructed from biodegradable suture, such as VICRYL™ (polyglactin 910) suture available from Ethicon Corp., Somerville, N.J.

The guide element 30 can be coupled to the self-assembling magnetic anastomosis device 16 with a number of different configurations and attachment mechanisms. The guide element 30 may be simply tied to the device 16, or the guide element 30 can be attached to the device 16 with an adhesive, e.g., acrylate glue, or with a fastener, such as a clip, screw, or rivet.

In some embodiments, a magnetic anastomosis device 16 may include multiple guide elements 30(1)-30(4). For example, as shown in FIGS. 8A, 8B, 8C, 8D, 8E, and 8F, a variety of attachment points can be used to provide control over the location and deployment of a self-assembling magnetic anastomosis device 16. As shown, four guide elements 30(1)-30(4) may be coupled to four separate segments of the device 16, respectively. Each guide element 30 may include a distal end coupled to a respective portion of the anastomosis device 16, and a proximal end that can be manipulated (i.e., increased or decreased tension) to thereby manipulate the positioning and orientation of the anastomosis device 16 once it has self-assembled into the pre-determined shape (i.e., a polygon). For example, as shown, guide element 30(1) is coupled to the most distal end segment, guide elements 30(2) and 30(3) are coupled to middle segments (segments between the most distal end segment and most proximal end segment), and guide element 30(4) is coupled to the most proximal end segment.

FIGS. 9A-9I illustrate delivery and placement of a first magnetic anastomosis device 16a at a target site and securing the first magnetic anastomosis device 16a to a first tissue 24 (i.e., distal tissue) via a securement apparatus 100 and subsequent delivery and placement of a second magnetic anastomosis device 16b at second tissue 22 (i.e., proximal tissue) at the target site and magnetic coupling of the first and second magnetic anastomosis devices 16a, 16b causing the first and second tissues 22, 26 to come together for subsequent formation of an anastomosis.

FIGS. 9A and 9B illustrate the formation of an enterotomy 63 in at least a first portion of the distal tissue 26 at the target site via a needle 32 or other piercing implement of the access device 14. The distal tissue 26 may include a bowel wall, for example. A surgeon may pass the access device into the GI tract, for example, and locate a target site (i.e., a site in which anastomosis formation is desired). As illustrated in FIG. 9B, upon piercing the tissue wall 26, the needle 32 may further be configured to anchor 34, or otherwise secure, the target site positioning. FIG. 9C illustrates delivery and self-assembling of the first magnetic anastomosis device 16a through the enterotomy 63 formed in the distal tissue wall 26 and into a lumen or cavity of the hollow body. As previously described, the device 16a is configured to self-assemble into the deployed configuration (i.e., a polygon shape, such as an octagon). As shown, the device 16a further includes guide elements 30.

FIGS. 9D-9G illustrates positioning of the first magnetic anastomosis device 16a via manipulation of the guide elements 30 coupled thereto and securing of the first magnetic anastomosis device 16a into place via the securement apparatus 100. For example, as shown in FIG. 9D, once the device 16a is fully deployed, the one or more guide elements 30 may generally extend from the anastomosis device 16a and through the enterotomy 63.

The securement apparatus 100 of the present invention is configured to secure the first magnetic anastomosis device 16a in place at the target site, thereby ensuring that the first magnetic anastomosis device 16a remains in position relative to the target site while the surgeon delivers and deploys of the second magnetic anastomosis device 16b at the target site. For example, as shown in FIG. 9E, the securement apparatus 100 generally includes a button member 101 including a body 103 having proximal end 102, an opposing distal end 104, and a central aperture 106 or through hole extending from the distal end 104 to the proximal end 102. The aperture 106 is shaped and/or sized to receive the one or more guide elements 30 therethrough, such that the button member 101 can generally translate along the length of the guide elements 30 extending from the first magnetic anastomosis device 16a and through the enterotomy 63, advancing the securement apparatus 100 towards the tissue 22. For example, once the first magnetic anastomosis device 16a is deployed at the target site (i.e., passed through an enterotomy 63 formed through a first tissue 26 at the target site and self-assembled into the predefined shape), the guide elements 30, which extend back through the enterotomy 63, can be threaded through the central aperture 106 of the body 103 of the button member 101. A surgeon then need only pull the guide elements 30 taught so as to draw the deployed first magnetic anastomosis device 16a into engagement with a first side of the distal tissue 26, essentially centering the first magnetic anastomosis device 16a relative to the enterotomy 63 and securing the first magnetic device 16a against the distal tissue 26, as shown in FIG. 9E. The button member 101 may then translate along a length of the guide elements 30, which pass through the central aperture 106, until the distal end 104 of the body engages an opposing side of the distal tissue 26, such that the button member 101 is centered relative to the enterotomy 63 and the opposing first magnetic anastomosis device 16a.

As shown in FIGS. 9F and 9G, the securement apparatus 100 further includes a plug member 108 including a distal end shaped and/or sized to be received within at least a proximal portion of the central aperture 106 of the button member 101 and retained therein such as via a friction fit, screw fit, snap fit, etc.

For example, the plug member 108 may taper in width from a proximal end to the distal end (i.e., greater width at proximal end and less width at the distal end). Accordingly, upon pulling the guide elements 30 taught and positioning the button member 101 into engagement with the first tissue 26, a surgeon can drive the plug member 108 into the central aperture 106 of the button member 101, which essentially secures the button member 101 and the opposing first magnetic anastomosis device 16a in position relative to the target site (i.e., fixed on opposing sides of the distal tissue wall 26).

More specifically, the plug member 108 secures the button member 101 and guide elements 30, which are passing through the central aperture 106 of the button member 101, to one another via the friction fit. The guide elements 30 can be cut and removed once the securement apparatus 100 and the opposing first magnetic anastomosis device 16a are essentially fixed in place along the tissue wall 26.

Upon fixing the first magnetic anastomosis device 16a in place, via the securement apparatus 100, the second magnetic anastomosis device 16b can be subsequently delivered and deployed at the target site via the access device 14, as shown in FIGS. 9H and 9I. Upon reaching the target tissue site, the first and second magnetic anastomosis devices 16a, 16b will be magnetically attracted to one another through a defined tissue area of the combined thickness the walls of the proximal 22 and distal 26 tissues at the target site (i.e., gallbladder 62 wall and stomach 60 wall, upper and lower intestinal walls, etc.) and exert compressive forces on the defined area to form the anastomosis. It should be noted that the button member 101 of the securement apparatus 100 is shaped and/or sized to be positioned between the first and second magnetic anastomosis devices 16a, 16b, and remain positioned therebetween, once the devices 16a, 16b have become magnetically attracted and coupled to one another. In particular, the button member 101 has a diameter that is less than the interior diameter of the polygon shape of each anastomosis device 16a, 16b when in a deployed configuration, such that the button member 101 does not interfere with magnetic coupling between corresponding segments of the first and second magnetic anastomosis devices 16a, 16b. However, the diameter of the button member 101 is greater than the diameter of the enterotomy 63. Accordingly, the button member 101 essentially remains fixed in position within the defined area of tissue to necrose and will subsequently be expelled once the anastomosis has formed. Furthermore, the button member 101 may include a contour that further prevents or reduces likelihood of interference with magnetic coupling between the devices 16a, 16b. For example, at least the proximal end 102 of the button member 101 may have a slope or dome-like shape.

Accordingly, the securement apparatus 100 of the present disclosure provides improved placement and securing of at least a first magnetic compression device 16a at a desired target site to thereby improve subsequent positioning and placement of a second corresponding magnetic anastomosis device 16b at the target site, so as to create accurate anastomoses between tissues. The securement apparatus 100 is configured to utilize guide elements 30 from a first anastomosis device 16a, already deployed and placed at the desired target tissue site, as a means for essentially fixing the first magnetic anastomosis device 16a in place, thereby ensuring a high degree of precision and improved placement of a subsequent second corresponding magnetic anastomosis device 16b to be coupled to the first device 16a (i.e., ensuring that the anastomosis devices are placed and coupled to one another at the intended target site) to achieve optimal placement and anastomosis formation.

Additional exemplary embodiments include an apparatus comprising a deployable self-assembling securement mechanism consisting of a body that holds the position of a compression anastomosis 16 device positioned at a target site relative to a first tissue wall 26 of a hollow body. The securement member captures the enterotomy 63 from the side opposite the compression anastomosis device 16a and holds the position until a second compression anastomosis device 16b can be mated to the locked device 16a. The securement member may also rotate along a central axis to manipulate an anastomosis device in order to align the poles of a pair of anastomosis devices.

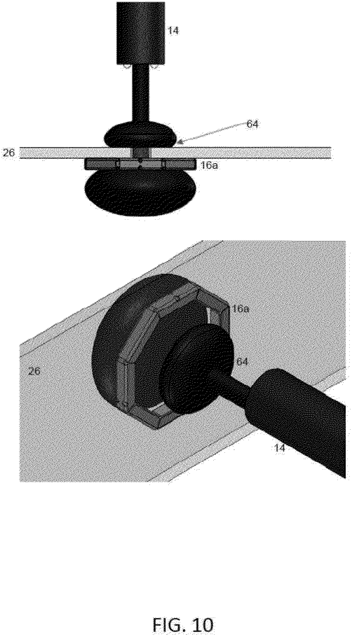

FIG. 10 shows a balloon securement mechanism 64 that can be inflated from the side of the enterotomy 63 opposite the compression anastomosis device 16a, in accordance with one exemplary embodiment. Upon deployment of the first magnetic anastomosis device 16a, the balloon securement mechanism 64 is deployed through the created enterotomy 63. Having been in a deflated/compressed position while stored within the access device 14, the balloon mechanism 64 is inflated to a deployed configuration as shown in FIG. 10. The balloon securement mechanism 64 engages with the distal anastomosis device 16a and holds the anastomosis device 16a in place against the first tissue wall 26 ensuring precise placement of the resulting anastomosis. In some alternative embodiments, the securement mechanism 64 may hold the anastomosis device 16a against both tissue walls 22, 26. The securement mechanism 64 may remain inside the patient after the access device 14 is removed. The securement mechanism 64 and the anastomosis devices 16a, 16b fall away from the formed anastomosis after time and are either removed from the patient or pass naturally.

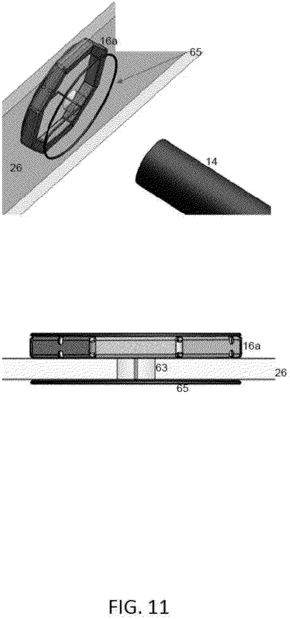

FIG. 11 shows a circular securement mechanism 65 that can self-deploy from the side of the enterotomy 63 opposite the compression anastomosis device 16a, in accordance with one exemplary embodiment. Upon deployment of the first magnetic anastomosis device 16a, the circular securement mechanism 65 is deployed through the created enterotomy 63. Having been stored in a compressed position within the access device 14 the circular securement mechanism 65 expands to a deployed configuration as shown in FIG. 11 due to being made of a shape memory material. The circular securement mechanism 65 engages with the distal magnetic anastomosis device 16a and holds the anastomosis device 16a in place against the first tissue wall 26 ensuring precise placement of the resulting anastomosis. In some alternative embodiments, the securement mechanism 64 may hold the anastomosis device 16a against both tissue walls 22, 26. The securement mechanism 65 may remain inside the patient after the access device 14 is removed. The securement mechanism 65 and the anastomosis devices 16a, 16b fall away from the formed anastomosis after time and are either removed from the patient or pass naturally.

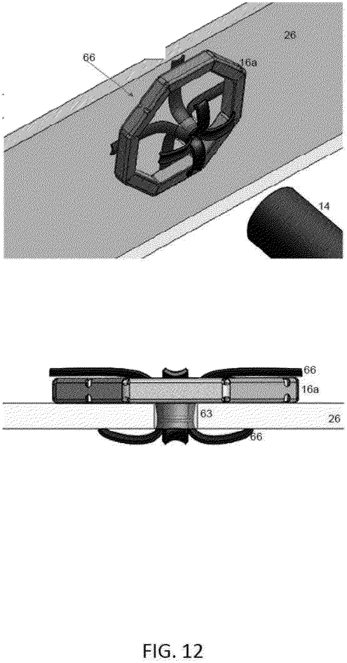

FIG. 12 shows a “flower petal” securement mechanism 66 including a plurality of curved members or springs (e.g., two, three, four, or more, which, for example, may be formed of a shape memory material or other material) that can be deployed from the side of the enterotomy 63 opposite the compression anastomosis device 16a, in accordance with one exemplary embodiment. The “flower petal” securement mechanism 66 is stored within the access device 14 in a compressed position. Upon delivery of the distal anastomosis device 16a, the “flower petal” securement mechanism 66 is deployed through the created enterotomy 63. As it is deployed, the “flower petal” securement apparatus 66 expands into its deployed configuration as shown in FIG. 12 due to being made of a shape memory material. The “flower petal” securement mechanism 66 engages with the distal anastomosis device 16a and holds the anastomosis device 16a in place against the first tissue wall 26 ensuring precise placement of the resulting anastomosis. In some alternative embodiments, the securement mechanism 66 may hold the anastomosis device 16a against both tissue walls 22, 26. The securement mechanism 65 may remain inside the patient after the access device 14 is removed. The securement mechanism 66 and the anastomosis devices 16a, 16b fall away from the formed anastomosis after time and are either removed from the patient or pass naturally.

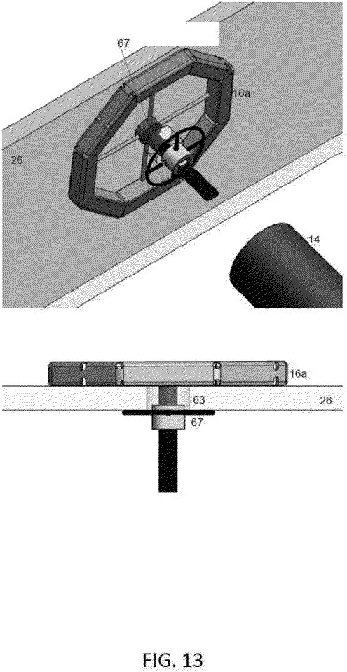

FIG. 13 shows a securement mechanism that can be deployed from the side of the enterotomy opposite the compression anastomosis device and also including a ratcheting mechanism 67 that only allows movement in the compressive direction, preventing any increase in the distance between the compression anastomosis device 16a and the securement mechanism 67, in accordance with one exemplary embodiment. Upon deployment of the distal anastomosis device 16a, the ratcheting securement mechanism 67 is deployed through the created enterotomy 63. Having been stored in a compressed configuration within the access device 14, the ratcheting securement mechanism 67 expands to its deployed configuration as shown in FIG. 13. The ratcheting securement mechanism 67 engages with the distal anastomosis device 16a and holds the anastomosis device in place against the first tissue wall 26. As the user pulls back on the access device 14, the ratcheting mechanism of the ratcheting securement mechanism 67 further compresses the anastomosis device 16a against the tissue wall 26, only allowing for movement in the compressive direction. In some alternative embodiments, the securement mechanism 67 may hold the anastomosis device 16a against both tissue walls 22, 26. The securement mechanism 67 may remain inside the patient after the access device 14 is removed. The securement mechanism 67 and the anastomosis devices 16a, 16b fall away from the formed anastomosis after time and are either removed from the patient or pass naturally.

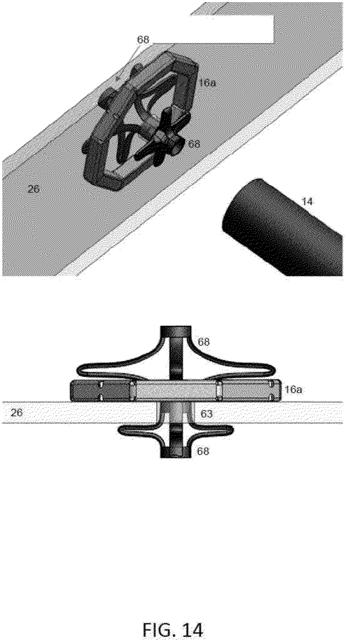

FIG. 14 shows a bulge tube securement mechanism 68 that expands on both sides of the enterotomy 63 by pulling the two ends toward one another and thereby to secure the compression anastomosis device 16a at the site of the enterotomy 63, in accordance with one exemplary embodiment. Upon deployment of the distal anastomosis device 16a, the bulge tube securement mechanism 68 is deployed through the created enterotomy 63. Having been stored in a compressed configuration while inside the access device 14, the bulge tube securement mechanism 68 expands as the user pulls back on the access device 14, bringing the two ends of the mechanism 68 toward one another into the deployed configuration as shown in FIG. 14. The bulge tube securement mechanism 68 may also auto-assemble into the deployed configuration due to being made of a shape memory material. The bulge tube securement mechanism 68 engages with the distal anastomosis device 16a and holds the anastomosis device 16a in place against the first tissue wall 26, ensuring precise placement of the resulting anastomosis. In some alternative embodiments, the securement mechanism 68 may hold the anastomosis device 16a against both tissue walls 22, 26. The securement mechanism 68 may remain inside the patient after the access device 14 is removed. The securement mechanism 68 and the anastomosis devices 16a, 16b fall away from the formed anastomosis after time and are either removed from the patient or pass naturally.

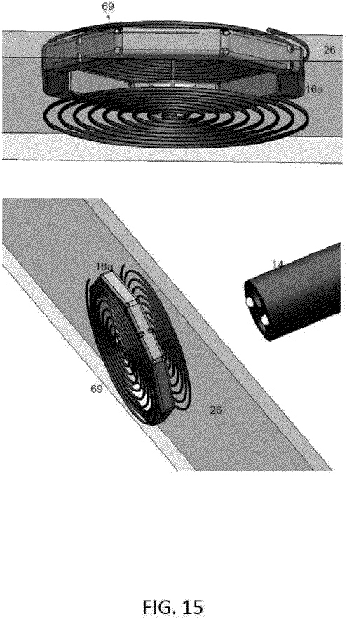

FIG. 15 shows a spiral spring securement mechanism 69 that can deployed from the side of the enterotomy 63 opposite the compression anastomosis device 16a, in accordance with one exemplary embodiment. Upon deployment of the distal anastomosis device 16a, the spiral spring securement mechanism 69 is deployed through the created enterotomy 63. Having been stored in a compressed configuration, within the access device 14, the spiral spring securement mechanism expands into its deployed configuration as shown in FIG. 15, due to being made of a shape memory material. The spiral spring securement mechanism 69 engages with the distal anastomosis device 16a and holds the anastomosis device 16a in place against the first tissue wall 26, ensuring precise placement of the resulting anastomosis. In some alternative embodiments, the securement mechanism 69 may hold the anastomosis device 16a against both tissue walls 22, 26. The securement mechanism 69 may remain inside the patient after the access device 14 is removed. The securement mechanism 69 and the anastomosis devices 16a, 16b fall away from the formed anastomosis after time and are either removed from the patient or pass naturally.

Of course, other types of securement mechanisms are possible in various alternative embodiments. It should be noted that any of the securement mechanisms can include a ratcheting mechanism that only allows movement in the compressive direction, preventing any increase in the distance between the compression anastomosis device and the securement mechanism.

Additional exemplary embodiments include an apparatus comprising a limiting member that sets the maximum separation distance between two compression anastomosis devices. The limiting member is rigidly attached to and/or contacts a compression anastomosis device 16a and accepts a connecting member from and/or attaches to a second compression anastomosis device 16b. The limiting member in any form will only allow movement in the compressive direction, preventing any increase in the distance between two compression anastomosis devices 16a, 16b. The limiting member may also rotate along a central axis to manipulate an anastomosis device in order to align the poles of a pair of anastomosis devices.

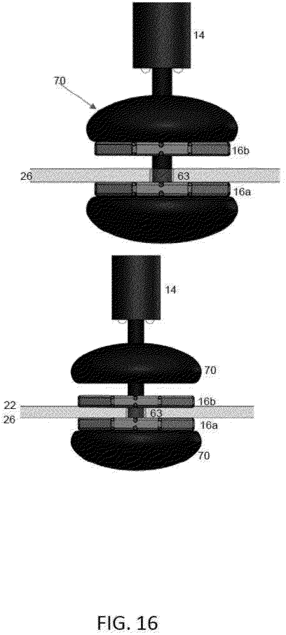

FIG. 16 shows a balloon limiting mechanism 70, in accordance with one exemplary embodiment. Upon deployment of the distal and proximal anastomosis devices 16a, 16b, the balloon limiting mechanism 70 is deployed through the created enterotomy 63. Having been stored in a compressed/deflated configuration while stored in the access device 14, the balloon limiting mechanism expands/inflates into its deployed configuration as shown in FIG. 16. The balloon limiting mechanism 70 has a distal and proximal side, which engage with the distal 16a and proximal 16b anastomosis devices respectively. Once engaged, the balloon limiting mechanism 70 may only move in the compressive direction, bringing the anastomosis devices 16a, 16b closer together against the tissue walls 22, 26. This ensures precise placement of the resulting anastomosis. The limiting mechanism 70 and the anastomosis devices 16a, 16b fall away from the formed anastomosis after time and are either removed from the patient or pass naturally.

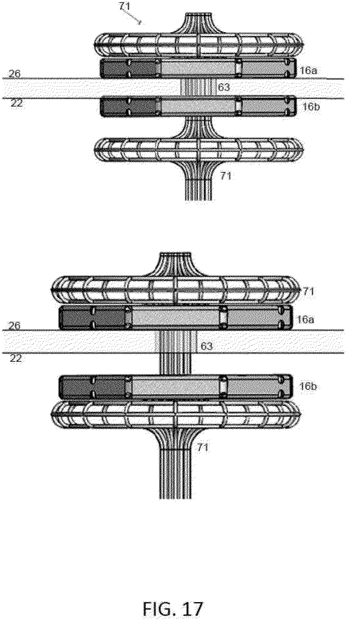

FIG. 17 shows a wire array limiting mechanism 71, in accordance with one exemplary embodiment. Upon deployment of the distal 16a and proximal 16b anastomosis devices, the wire array limiting mechanism 71 is deployed through the created enterotomy 63. Having been stored in a compressed configuration while inside the access device 14, the wire array limiting mechanism 71 expands into its deployed configuration as shown in FIG. 17 due to it being made of a shape memory material. The wire array limiting mechanism 71 has a distal and proximal end, which engage with the distal 16a and proximal 16b anastomosis devices respectively. Once engaged, the wire array limiting mechanism 71 may only move in the compressive direction, bringing the anastomosis devices 16a, 16b closer together against the tissue walls 22, 26. This ensures precise placement of the resulting anastomosis. The limiting mechanism 71 and the anastomosis devices 16a, 16b fall away from the formed anastomosis after time and are either removed from the patient or pass naturally.

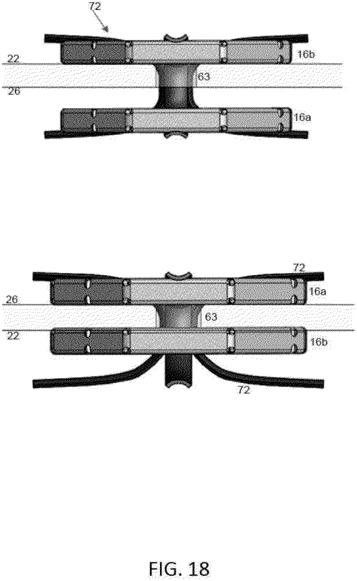

FIG. 18 shows a “flower petal” limiting mechanism 72 including a plurality of curved members or springs (e.g., two, three, four, or more, which, for example, may be formed of a shape memory material or other material), in accordance with one exemplary embodiment. Upon deployment of the distal 16a and proximal 16b anastomosis devices, the “flower petal” limiting mechanism 72 is deployed through the created enterotomy 63. Having been stored in a compressed configuration, the “flower petal” limiting mechanism 72 expands into its deployed configuration as shown in FIG. 18, due to it being made of a shape memory material. The “flower petal” limiting mechanism 72 has a distal and proximal end, which engage with the distal 16a and proximal 16b anastomosis devices respectively. Once engaged, the “flower petal” limiting mechanism 72 may only move in the compressive direction, bringing the anastomosis devices 16a, 16b closer together against the tissue walls 22, 26. This ensures precise placement of the resulting anastomosis. The limiting mechanism 72 and the anastomosis devices 16a, 16b fall away from the formed anastomosis after time and are either removed from the patient or pass naturally.

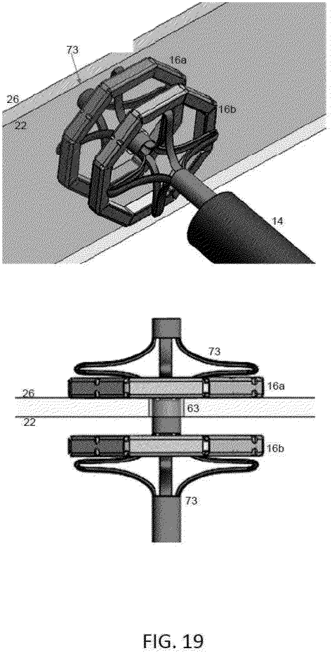

FIG. 19 shows a bulge tube limiting mechanism 73, in accordance with one exemplary embodiment. Upon deployment of the distal 16a and proximal 16b anastomosis devices, the bulge tube limiting mechanism is deployed through the created enterotomy 63. Having been stored in a compressed configuration while inside the access device 14, the bulge tube limiting mechanism 73 expands as the user pulls back on the access device 14, bringing the two ends of the mechanism 73 toward one another, into the deployed configuration as shown in FIG. 19. The bulge tube limiting mechanism may also auto-configure into the deployed configuration due to being made of a shape memory material. The bulge tube limiting mechanism 73 has a distal and proximal end that engage with the distal 16a and proximal 16b anastomosis devices respectively. Once engaged, the bulge tube limiting mechanism 73 may only move in the compressive direction, bringing the anastomosis devices 16a, 16b closer together against the tissue walls 22, 26. This ensures precise placement of the resulting anastomosis. The limiting mechanism 73 and the anastomosis devices 16a, 16b fall away from the formed anastomosis after time and are either removed from the patient or pass naturally.

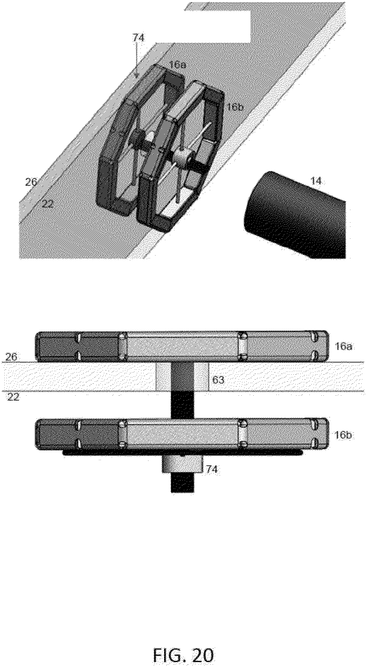

FIG. 20 shows a limiting mechanism that also includes a ratcheting mechanism 74 that only allows movement in the compressive direction, preventing any increase in the distance between the compression anastomosis device 16a and the securement mechanism 74, in accordance with one exemplary embodiment. Upon deployment of the distal 16a and proximal 16b anastomosis devices, the ratcheting limiting mechanism is deployed through the created enterotomy 63. Having been stored in a compressed position, the ratcheting limiting mechanism 74 expands into its deployed configuration as shown in FIG. 20 due to it being made of a shape memory material. The ratcheting limiting mechanism 74 has a distal and proximal end that engage with the distal 16a and proximal 16b anastomosis devices respectively. Once engaged, the ratcheting limiting mechanism may only move in the compressive direction due to a ratcheting mechanism on the device. As the user pulls back on the access device 14, the ratcheting mechanism ratchets the device in the compressive direction only, bringing the anastomosis devices 16a, 16b closer together against the tissue walls 22, 26. This ensures precise placement of the resulting anastomosis. The limiting mechanism 74 and the anastomosis devices 16a, 16b fall away from the formed anastomosis after time and are either removed from the patient or pass naturally.

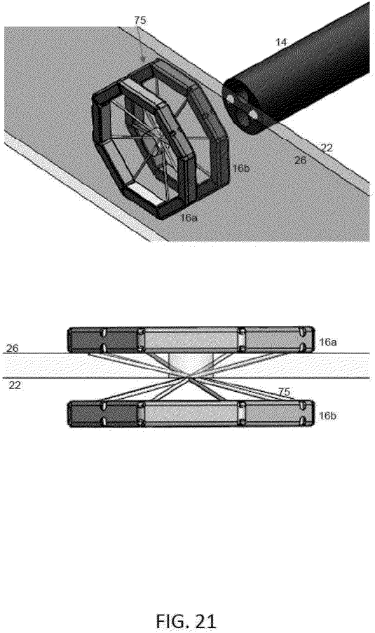

FIG. 21 shows a limiting mechanism with suspenders 75, in accordance with one exemplary embodiment. Upon deployment of the distal 16a and proximal 16b anastomosis devices, the suspender limiting mechanism 75 is deployed through the created enterotomy 63. Having been stored in a compressed position within the access device 14, the suspender limiting mechanism 75 expands to its deployed configuration as shown in FIG. 21 due to it being made of a shape memory material. The suspender limiting mechanism 75 has a distal and proximal end that engage with the distal 16a and proximal 16b anastomosis devices respectively. Once engaged, the suspender limiting mechanism 75 may only move in the compressive direction, bringing the anastomosis devices 16a, 16b closer together against the tissue walls 22, 26. This ensures precise placement of the resulting anastomosis. The limiting mechanism 75 and the anastomosis devices 16a, 16b fall away from the formed anastomosis after time and are either removed from the patient or pass naturally.

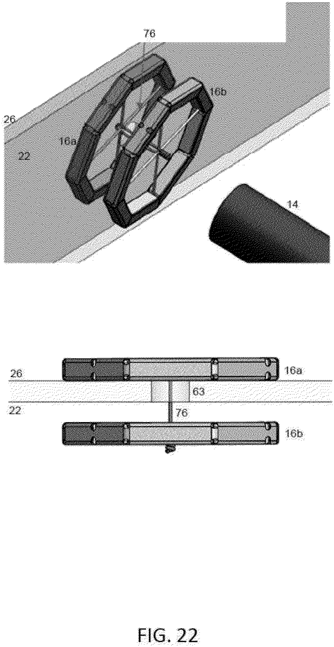

FIG. 22 shows a rebar tie limiting mechanism 76, in accordance with one exemplary embodiment. Upon deployment of the distal 16a and proximal 16b anastomosis devices, the rebar tie limiting mechanism 76 is deployed through the created enterotomy 63. Having been stored in a compressed configuration within the access device 14, the rebar tie limiting mechanism 76 expands to its deployed configuration as shown in FIG. 22 due to it being made of a shape memory material. The rebar tie limiting mechanism 76 has a distal and proximal end that engage with the distal 16a and proximal 16b anastomosis devices. Once engaged, the rebar tie limiting mechanism may only move in the compressive direction, bringing the anastomosis devices 16a, 16b closer together against the tissue walls 22, 26. This ensures precise placement of the resulting anastomosis. The limiting mechanism 76 and the anastomosis devices 16a, 16b fall away from the formed anastomosis after time and are either removed from the patient or pass naturally.

Of course, other types of limiting mechanisms are possible in various alternative embodiments. It should be noted that any of the limiting mechanisms can include a ratcheting mechanism that only allows movement in the compressive direction, preventing any increase in the distance between the compression anastomosis device and the securement mechanism.

### Incorporation by Reference

References and citations to other documents, such as patents, patent applications, patent publications, journals, books, papers, web contents, have been made throughout this disclosure. All such documents are hereby incorporated herein by reference in their entirety for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes which come within the meaning and range of equivalency of the claims are therefore intended to be embraced therein Potential Claims

Various embodiments of the present invention may be characterized by the potential claims listed in the paragraphs following this paragraph (and before the actual claims provided at the end of the application). These potential claims form a part of the written description of the application. Accordingly, subject matter of the following potential claims may be presented as actual claims in later proceedings involving this application or any application claiming priority based on this application. Inclusion of such potential claims should not be construed to mean that the actual claims do not cover the subject matter of the potential claims. Thus, a decision to not present these potential claims in later proceedings should not be construed as a donation of the subject matter to the public. Nor are these potential claims intended to limit various pursued claims.

Without limitation, potential subject matter that may be claimed (prefaced with the letter “P” so as to avoid confusion with the actual claims presented below) includes:

P1. A securement apparatus for assisting in placing and securing a magnetic compression device at a desired target site within a patient, the securement apparatus comprising:
a button member comprising a body including a proximal end, an opposing distal end, and a central aperture extending entirely therethrough, wherein the aperture is shaped and/or sized to receive and allow one or more guide elements, coupled to a first magnetic compression device positioned at a target site relative to a first tissue wall of a hollow body of the patient, to pass therethrough, wherein the button member is configured to translate along a length of the one or more guide elements towards the target site within a hollow body of the patient; and
a plug member comprising a distal end shaped and/or sized to be received within at least a proximal portion of the central aperture of the button member and retained therein via a friction fit to thereby secure the button member at a position along the length of the one or more guide elements.

P2. The securement apparatus of claim P1, wherein the proximal end of the button member comprises an arcuate contour.

P3. The securement apparatus of claim P2, wherein the proximal end of the button member has a dome shape.

P4. The securement apparatus of claim P1, wherein the distal end of the button member is configured to engage a portion of the first tissue wall of the hollow body of the patient.

P5. The securement apparatus of claim P4, wherein, upon securing the button member at a position along the length of the one or more guide elements via positioning of the plug member within the central aperture of the button member, the first magnetic compression device is secured at a position relative to the first tissue wall of the hollow body of the patient.

P6. The securement apparatus of claim P5, wherein the button member and the first magnetic compression device are coaxially aligned with one another when the button member secured at a position along the length of the one or more guide elements via positioning of the plug member within the central aperture of the button member.

P7. The securement apparatus of claim P1, wherein the button member comprises a diameter that is less than an interior diameter of the first magnetic compression device when the first magnetic compression device is in a fully deployed configuration.

P8. The securement apparatus of claim P1, wherein the button member comprises a diameter that is greater than a diameter of an opening formed in the first tissue wall through which the one or more guide elements pass.

P9. The securement apparatus of claim P1, wherein the plug member comprises a proximal end opposing the distal end.

P10. The securement apparatus of claim P9, wherein the distal end of the plug member comprises a shape and/or contour complementary to a shape and/or contour of at least the proximal portion of the central aperture of the button member.

P11. The securement apparatus of claim P10, wherein the plug member tapers in width from the proximal end towards the distal end.

P12. The securement apparatus of claim P11, wherein at least the proximal portion of the central aperture tapers in width from a proximal-most opening of the central aperture adjacent to the proximal end of the button member towards a distal-most opening of the central aperture adjacent to the distal end of the button member.

P13. The securement apparatus of claim P10, wherein a cross-sectional shape of the distal end of the plug member corresponds to and complements a cross-sectional shape of at least the proximal portion of the central aperture of the button member.

P14. The securement apparatus of claim P1, wherein each of the button member and plug member comprises a medical grade material.

P15. The securement apparatus of claim P1, wherein the securement apparatus is shaped and/or sized to be endoscopically introduced into the patient.

P16. The securement apparatus of claim P1, wherein the securement apparatus is shaped and/or sized to be laparoscopically introduced into the patient.

P17. An apparatus comprising a deployable self-assembling securement member consisting of a body that holds the position of a compression anastomosis device positioned at a target site relative to a first tissue wall of a hollow body. The securement member captures the enterotomy and holds the position until a second compression anastomosis device can be mated to the locked device.

P18. The apparatus of claim P17, wherein the securement member is a balloon securement member.

P19. The apparatus of claim P17, wherein the securement member is a circular securement member.

P20. The apparatus of claim P17, wherein the securement member is a “flower petal” securement member.

P21. The apparatus of claim P17, wherein the securement member is a ratcheting securement member.

P22. The apparatus of claim P17, wherein the securement member is a bulge tube securement member.

P23. The apparatus of claim P17, wherein the securement member is a spiral spring securement member.

P24. An apparatus comprising a limiting member that sets the maximum separation distance between two compression anastomosis devices. The limiting member is rigidly attached to and/or contacts a compression anastomosis device and accepts a connecting member from and/or attaches to a second compression anastomosis device. The limiting member in any form will only allow movement in the compressive direction, preventing any increase in the distance between two compression anastomosis devices.

P25. The apparatus of claim P24, wherein the limiting member is a balloon limiting member.

P26. The apparatus of claim P24, wherein the limiting member is a wire array limiting member.

P27. The apparatus of claim P24, wherein the limiting member is a “flower petal” limiting member.

P28. The apparatus of claim P24, wherein the limiting member is a bulge tube limiting member.

P29. The apparatus of claim P24, wherein the limiting member is a ratcheting limiting member.

P30. The apparatus of claim P24, wherein the limiting member include suspenders.

P31. The apparatus of claim P24, wherein the limiting member is a rebar tie limiting member.

What is claimed is:

1. A securement apparatus for assisting in placing and securing an anastomosis device at a desired target site within a patient, the securement apparatus comprising:
a button member comprising a body having a proximal end, a distal end, and a central aperture extending entirely therethrough, wherein the aperture is shaped and/or sized to receive one or more guide elements therethrough to allow coupling of the one or more guide elements to a first distal anastomosis device at a target site, wherein the button member is capable of translating along the one or more guide elements toward the target site within the patient to position the button member relative to an enterotomy and the first anastomosis device; and
a plug member comprising a distal end and an opposing proximal end, wherein the distal end of the plug member is shaped and/or sized to be received within at least the proximal portion of the central aperture of the button member to be retained therein to secure the one or more guide elements and the first anastomosis device in place against a tissue and/or organ wall.

2. The securement apparatus of claim 1, wherein the plug member is tapered in width such that the proximal end is wider than the distal end.

3. The securement apparatus of claim 1, wherein the button member is shaped and/or sized to be positioned between the first anastomosis device and a second anastomosis device.

4. The securement apparatus of claim 1, wherein the diameter of the button member is larger than that of the enterotomy.

5. The securement apparatus of claim 1, wherein the proximal end has a sloped shape.

6. The securement apparatus of claim 1, wherein the proximal end has a dome shape.

7. The securement apparatus of claim 1, wherein the distal end of the button member is configured to engage a portion of the first tissue wall of the hollow body of the patient.

8. The securement apparatus of claim 2, wherein at least the proximal portion of the central aperture tapers in width from a proximal-most opening of the central aperture adjacent to the proximal end of the button member towards a distal-most opening of the central aperture adjacent to the distal end of the button member.

9. The securement apparatus of claim 1, wherein each of the button member and plug member comprises a medical grade material.

10. The securement apparatus of claim 2, wherein the button member is shaped and/or sized to be positioned between the first anastomosis device and a second anastomosis device.

11. The securement apparatus of claim 2, wherein the diameter of the button member is larger than that of the enterotomy.

12. The securement apparatus of claim 2, wherein the proximal end has a sloped shape.

13. The securement apparatus of claim 2, wherein the proximal end has a dome shape.

14. The securement apparatus of claim 2, wherein the distal end of the button member is configured to engage a portion of the first tissue wall of the hollow body of the patient.

15. The securement apparatus of claim 2, wherein each of the button member and plug member comprises a medical grade material.

16. The securement apparatus of claim 1, wherein the plug secures the one or more guides and also prevents the button from moving along the guides away from the anastomosis when the plug member is retained in the button member.

17. The securement apparatus of claim 2, wherein the plug secures the one or more guides and also prevents the button from moving along the guides away from the anastomosis when the plug member is retained in the button member.

18. The securement apparatus of claim 1, wherein the button member is centered relative to the enterotomy and the first anastomosis device.

19. The securement apparatus of claim 1, wherein the guide elements are guidewires.

20. The securement apparatus of claim 1, wherein the plug member is retained in the central aperture via a friction fit.

21. A method of securing a compression anastomosis device at a desired target site within a patient using a securement apparatus according to claim 1, the method comprising:
delivering the first compression anastomosis device through an enterotomy to a target site within the patient at a distal side of a tissue and/or organ wall, wherein the first compression anastomosis device is coupled to one or more guide elements that pass through the enterotomy and through the central aperture of the button member with the distal end of the button member oriented toward the enterotomy;
translating the button member along the one or more guide elements toward the target site within the patient to position the button member relative to the enterotomy and the first anastomosis device such that the distal end of the button member engages with a proximal side of the tissue and/or organ wall opposite the first anastomosis device; and
inserting the plug member into the central aperture of the button member to secure the one or more guide elements with the button member and the first anastomosis device respectively secured against the proximal and distal sides of the tissue and/or organ wall at the target site.
